(19) United States
(12) Patent Application Publication
(10) Pub. No.: US 2005/0009775 A1
(43) Pub. Date: Jan. 13, 2005

(54) Nucleoside compounds in hcv

(76) Inventors: Peter David Howes, Stevenage (GB); Martin John Slater, Stevenage (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/481,081

(22) PCT Filed: May 15, 2002
(86) PCT No.: PCT/GB02/02269

(30) Foreign Application Priority Data
Jun. 21, 2001 (GB) 0115222.2
Jan. 15, 2002 (GB) 0200832.4

(51) Int. Cl. C07H 19/04; A61K 31/7076; A61K 31/7072

(52) U.S. Cl. 514/45; 514/46; 514/50; 536/26.1

(57) ABSTRACT

Protide compounds of formula (I) 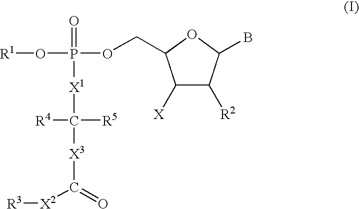
wherein X represents H, F, N₃, NH₂, —CN, or —OMe; X¹ represents O or NR⁷; X² represents O, NH, NR⁶ or S, or when X³ is O then X² is absent; X³ is absent, or when X¹ is O then X³ represents O; R¹ represents hydrogen; optionally substituted C₁₋₆alkyl; optionally substituted aryl; or optionally substituted heteroaryl; R² represents hydroxy, OCOR⁶, or OCO₂R⁶; R³ represents H, optionally substituted C₁₋₆alkyl, optionally substituted aryl, optionally substituted heteroaryl or optionally substituted heterocyclyl; R⁴ and R⁵ are independently selected from hydrogen, optionally substituted C₁₋₆alkyl, optionally substituted aryl, or optionally substituted aralkyl; R⁶ represents optionally substituted C₁₋₆alkyl or optionally substituted aryl; R⁷ represents H, optionally substituted C₁₋₆alkyl, or optionally substituted aryl, wherein when R⁴ and R⁷ are each alkyl they may be linked to form a 5- or 6-membered ring;
B represents (a), (b), (c), or (d) 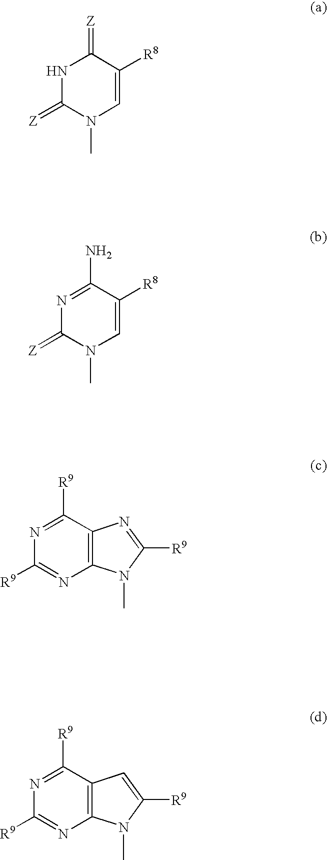
wherein Z represents O or S; R⁸ represents H, halo, C₂₋₄alkynyl, trifluoromethyl, C₁₋₃alkoxy, hydroxy, methylthio, amino, nitro, or C₁₋₃alkyl wherein the C₁₋₃alkyl may be optionally substituted by hydroxy, halo, amino, or OR¹⁰ wherein R¹⁰ represents C₁₋₆alkyl optionally substituted by aryl which may itself be optionally substituted; and R⁹ represents H, halo, hydroxy, OR⁶, SR⁶ or NR³R³; are useful in the treatment of viral infection, particularly HCV infection.

### FIELD OF TE INVENTION

The present invention relates to protide derivatives of therapeutically active nucleoside derivatives, processes for their manufacture, pharmaceutical formulations comprising them and their use in therapy, particularly for the treatment or prophylaxis of certain viral infections. In particular, we have found a group of compounds that are useful in treating viral infections, especially hepatitis C virus (HCV) infection.

### BACKGROUND OF THE INVENTION

In the US, an estimated 4.5 million Americans are chronically infected with HCV.

Although only 30% of acute infections are symptomatic, greater than 85% of infected individuals develop chronic, persistent infection. Treatment costs for HCV infection have been estimated at $5.46 billion for the US in 1997. Worldwide over 200 million people are estimated to be infected chronically. HCV infection is responsible for 40-60% of all chronic liver disease and 30% of all liver transplants. Chronic HCV infection accounts for 30% of all cirrhosis, end-stage liver disease, and liver cancer in the U.S. The CDC estimates that the number of deaths due to HCV will minimally increase to 38,000/year by the year 2010.

Due to the high degree of variability in the viral surface antigens, existence of multiple viral genotypes, and demonstrated specificity of immunity, the development of a successful vaccine in the near future is unlikely. Alpha-interferon (alone or in combiation with ribavirin) has been widely used since its approval for treatment of chronic HCV infection. However, adverse side effects are commonly associated with this treatment: flu-like symptoms, leukopenia, thrombocytopenia, depression from interferon, as well as anemia induced by ribavirin (Lindsay, K. L. (1997) Hepatology 26 (suppl 1):71S-77S). This therapy remains less effective against infections caused by HCV genotype 1 (which constitutes ˜75% of all HCV infections in the developed markets) compared to infections caused by the other 5 major HCV genotypes. Unfortunately, only ˜50-80% of the patients respond to this treatment (measured by a reduction in serum HCV RNA levels and normalization of liver enzymes) and, of those treated, 50-70% relapse within 6 months of cessation of treatment. Recently, with the introduction of pegylated interferon, both initial and sustained response rates have improved substantially, and combination treatment of Peg-IFN with ribavirin constitutes the gold standard for therapy. However, the side effects asociated with combination therapy and the impaired response in patients with genotype 1 present opportunities for improvement in the management of this disease.

First identified by molecular cloning in 1989 (Choo, Q-L et al (1989) Science 244:359-362), hepatitis C virus (HCV) is now widely accepted as the most common causative agent of post-transfusion non A, non-B hepatitis (NANBH) (Kuo, G et al (1989) Science 244:362-364). Due to its genome structure and sequence homology, this virus was assigned as a new genus in the Flaviviridae family. Like the other members of the Flaviviridae, such as flaviviruses (e.g. yellow fever virus and Dengue virus types 1-4) and pestiviruses (e.g. bovine viral diarrhea virus, border disease virus, and classic swine fever virus) (Choo, Q-L et al (1989) Science 244:359-3; Miller, R. H. and R. H. Purcell (1990) Proc. Natl. Acad. Sci. USA 87:2057-2061), HCV is an enveloped virus containing a single strand RNA molecule of positive polarity. The HCV genome is approximately 9.6 kilobases (kb) with a long, highly conserved, noncapped 5′ nontranslated region (NTR) of approximately 340 bases which functions as an internal ribosome entry site (IRES) (Wang CY et al ‘An RNA pseudoknot is an essential structural element of the internal ribosome entry site located within the hepatitis C virus 5′ noncoding region’ [Article] Rna-A Publication of the Rna Society. 1(5):526-537, 1995 July). This element is followed by a region which encodes a single long open reading frame (ORF) encoding a polypeptide of ˜3000 amino acids comprising both the structural and nonstructural viral proteins.

Upon entry into the cytoplasm of the cell, this RNA is directly translated into a polypeptide of ˜3000 amino acids comprising both the structural and nonstructural viral proteins. This large polypeptide is subsequently processed into the individual structural and nonstructural proteins by a combination of host and virally-encoded proteinases (Rice, C. M. (1996) in B. N. Fields, D. M. Knipe and P. M. Howley (eds) Virology 2^{nd} Edition, p931-960; Raven Press, N.Y.). Following the termination codon at the end of the long ORF, there is a 3′ NTR which roughly consists of three regions: an 40 base region which is poorly conserved among various genotypes, a variable length poly(U)/polypyrimidine tract, and a highly conserved 98 base element also called the “3′X-tail” (Kolykhalov, A. et al (1996) J. Virology 70:3363-3371; Tanaka, T. et al (1995) Biochem Biophys. Res. Commun. 215:744-749; Tanaka, T. et al (1996) J. Virology 70:3307-3312; Yarnada, N. et al (1996) Virology 223:255-261). The 3′ NTR is predicted to form a stable secondary structure which is essential for HCV growth in chimps and is believed to function in the initiation and regulation of viral RNA replication.

The NS5B protein (591 amino acids, 65 kDa) of HCV (Behrens, S. E. et al (1996) EMBO J. 15:12-22), encodes an RNA-dependent RNA polymerase (RdRp) activity and contains canonical motifs present in other RNA viral polymerases. The NS5B protein is fairly well conserved both intra-typically (95-98% amino acid (aa) identity across lb isolates) and inter-typically (˜85% aa identity between genotype 1a and 1b isolates). The essentiality of the HCV NS5B RdRp activity for the generation of infectious progeny virions has been formally proven in chimpanzees (A. A. Kolylhalov et al. (2000) Journal of Virology, 74(4), p.2046-2051). Thus, inhibition of NS5B RdRp activity (inhibition of RNA replication) is predicted to cure HCV infection.

Based on the foregoing, there exists a significant need to identify synthetic or biological compounds for their ability to inhibit HCV.

Some nucleoside derivatives, for example AZT, 3TC and abacavir, which are useful in the treatment of HIV, are known to undergo metabolism when inside cells to form phosphate derivatives. In particular, triphosphate derivatives have been demonstrated to be active against some viral targets. However, triphosphate compounds are not easily transported across cell membranes so that they are often not suitable for direct administration to patients.

It has now been discovered that derivatives of certain nucleoside compounds have potential for the treatment or prophylaxis of viral infections, for example hepatitis C virus, due to their ability to inhibit HCV polymerase or their ability to gain entry to cells where they are converted to compounds which inhibit HCV polymerase.

### DETAILED DESCRIPTION OF INVENTION

According to one aspect of the present invention, we provide compounds of formula (I) 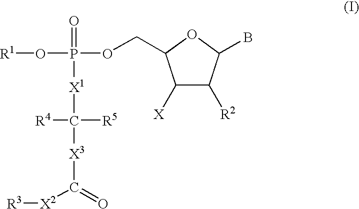
wherein X represents H, F, N₃, NH₂, —CN, or —OMe;
X¹ represents O or NR⁷;
X² represents O, NH, NR⁶ or S, or when X³ is O then x² is absent;
X³ is absent, or when X¹ is O then X³ represents O;
R¹ represents hydrogen; optionally substituted C₁₋₆alkyl; optionally substituted aryl; or optionally substituted heteroaryl;
R² represents hydroxy, OCOR⁶, or OCO₂R⁶;
R³ represents H, optionally substituted C₁₋₆alkyl, optionally substituted aryl, optionally substituted heteroaryl or optionally substituted heterocyclyl;
R⁴ and R⁵ are independently selected from hydrogen, optionally substituted C₁₋₆alkyl, optionally substituted aryl, or optionally substituted aralkyl;
R⁶ represents optionally substituted C₁₋₆alkyl or optionally substituted aryl;
R⁷ represents H, optionally substituted C₁₋₆alkyl, or optionally substituted aryl, wherein when R⁴ and R⁷ are each alkyl they may be linked to form a 5- or 6-membered ring;
B represents (a), (b), (c), or (d) 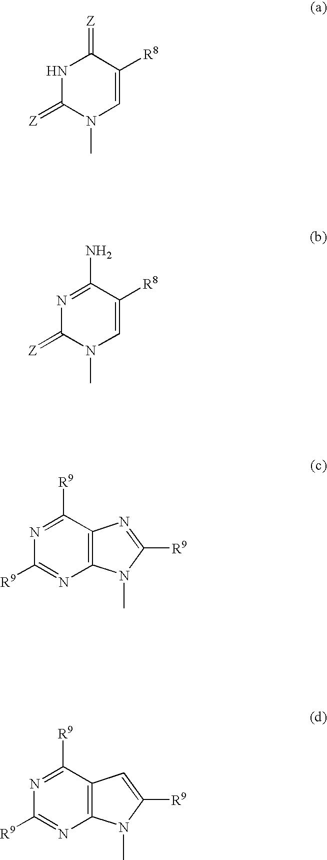
   wherein Z represents O or S;
R⁸ represents H, halo, C₂₋₄alkynyl, trifluoromethyl, C₁₋₃alkoxy, hydroxy, methylthio, amino, nitro, or C₁₋₃alkyl wherein the C₁₋₃alkyl may be optionally substituted by hydroxy, halo, amino, or OR¹⁰ wherein R′″ represents C₁₋₆alkyl optionally substituted by aryl which may itself be optionally substituted; and
R⁹ represents H, halo, hydroxy, OR⁶, SR⁶ or NR³R³; and salts and solvates thereof (hereinafter “compounds of the invention”).

According to a further aspect of the present invention, we provide compounds of formula (Ia) 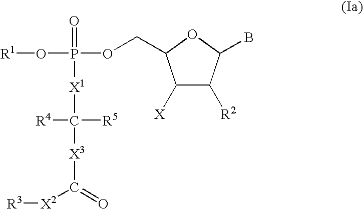
wherein X represents H, F, N₃, NH₂, —CN, or —OMe;
X¹ represents O or NR⁷;
X² represents O, NH, N⁶ or S, or when X³ is O then x² is absent;
X³ is absent, or when X¹ is O then X³ represents O;
R¹ represents hydrogen; optionally substituted aryl; or optionally substituted heteroaryl;
R² represents hydroxy, OCOR⁶, or OCO₂R⁶;
R³ represents H, optionally substituted C₁₋₆alkyl, optionally substituted aryl, optionally substituted heteroaryl or optionally substituted heterocyclyl;
R⁴ and R⁵ are independently selected from hydrogen, optionally substituted C₁₋₆alkyl, optionally substituted aryl, or optionally substituted aralkyl;
R⁶ represents optionally substituted C₁₋₆alkyl or optionally substituted aryl;
R⁷ represents H, optionally substituted C₁₋₆alkyl, or optionally substituted aryl, wherein when R⁴ and R⁷ are each alkyl they may be linked to form a 5- or 6-membered ring;
B represents (a), (b), (c), or (d) 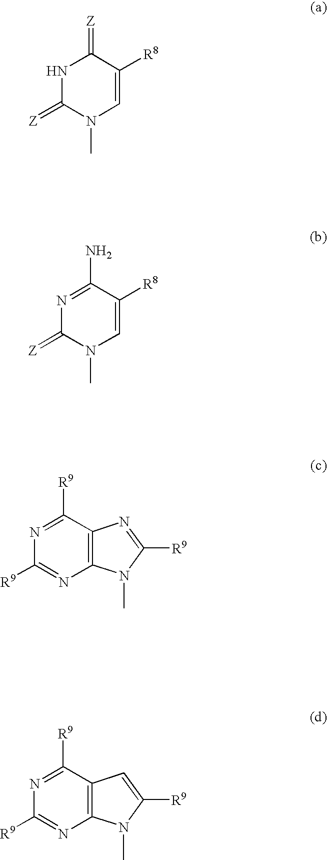
wherein Z represents O or S;
R⁸ represents halo, C₂₋₄alkynyl, trifluoromethyl, C₁₋₃alkoxy, hydroxy, methylthio, amino, nitro, or C₁₋₃alkyl wherein the C₁₋₃alkyl may be optionally substituted by hydroxy, halo, amino, or OR¹⁰ wherein R¹⁰ represents C₁₋₆alkyl optionally substituted by aryl which may itself be optionally substituted; and
R⁹ represents H, halo, hydroxy, OR⁶, SR⁶ or NR³R³;
and salts and solvates thereof.

Compounds of formula (I) and (Ia) contain more than one asymmetric carbon atom, and the invention includes all diastereoisomers of compounds of formula (I) and (Ia) and mixtures thereof.

The present invention also includes the physiologically acceptable salts of the compounds of formula (I) and (Ia). Suitable physiologically acceptable salts of the compounds of formula (I) and (Ia) include acid salts, for example sodium, potassium, calcium, magnesium and tetraalkylammonium and the like, or mono- or di-basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like.

The present invention also relates to solvates of the compounds of Formula (I) and (Ia), for example hydrates.

The present invention relates to protide compounds (prodrugs of nucleoside monophosphates), as defined by Formula (I) and (Ia). When used herein, the term “protide” refers to stabilized phosphate derivatives, for example such derivatives as described in Koszalka, G. W., Daluge, S. M., Boyd, F. L., Annual Rep Med Chem 1998, 33, 163-171 and the references cited therein (the contents of which are incorpoarted herein by reference thereto). Examples of protides include, but are not restricted to, phosphoramidates of the compounds of Formula (I) and (Ia).

When used herein, the term “phosphoramidate” refers to a group attached to the phosphorus atom of a mono-phosphate derivative (nucleotide) of Formula (I) and (Ia), via a nitrogen atom.

When used herein, the term “alkyl” includes a branched or unbranched, cyclic or acyclic, saturated or unsaturated (e.g. alkenyl or alkynyl) hydrocarbyl radical. The term “Me” means methyl. When used herein, the term “alkynyl” includes branched as well as straight chain alkynyl, for example ethynyl and propynyl.

When used herein, the term “aryl” represents an optionally substituted 5 to 14 membered, preferably 6 to 10 membered, monocylic or bicyclic aromatic ring system, for example phenyl.

When used herein, the term “heteroaryl” represents an optionally substituted 5 to 14 membered, preferably 6 to 10 membered, monocylic or bicyclic aromatic ring system, comprising one to four heteroatoms selected from O, N and S.

When used herein, the term “heterocyclyl” represents an optionally substituted, 5 or 6 membered, saturated cyclic hydrocarbon group containing one to four heteroatoms selected from N, optionally substituted by hydrogen, C₁₋₆alkyl, C(O)R³, SO₂R³, aryl or heteroaryl; O; and S, optionally substituted by one or two oxygen atoms.

When used herein, the term “halo” represents chloro, bromo, fluoro, or iodo.

Unless otherwise stated, when used herein, the term “optionally substituted” includes aryl (e.g. phenyl), C₁₋₆alkyl (e.g. methyl, ethyl), nitro, oxo, OR, CO₂R, SO₂R, NRR, CONRR, SONRR, CH₂N(Me)₂, and halo. R represents H, C₁₋₆alkyl, or aryl.
Preferably, X represents H;
Preferably X¹ represents NR⁷ where R⁷ represents H;
Preferably X² represents O;
Preferably X³ is absent;
Preferably R¹ represents optionally substituted aryl or optionally substituted heteroaryl; more preferably R¹ represents optionally substituted aryl; most preferably R¹ represents phenyl or 4-tert-butylphenyl;
Preferably R² represents hydroxy;
Preferably R³ represents optionally substituted C₁₋₆alkyl; more preferably R³ represents methyl or benzyl;
Preferably R⁴ represents H;
Preferably R⁵ represents C₁₋₆alkyl; more preferably R⁵ represents methyl;
Preferably B represents (b) or (c);
Preferably R⁸ represents H;
Preferably R⁹ represents H, hydroxy or NR³R³ where R³ represents H;
Preferably Z represents O;

Preferably the stereochemistry of the sugar is beta-D-ribofuranose.

Preferred compounds of Formula (I) include:
3′-deoxyguanosine 5′-[4-(1, 1-dimethylethyl)phenyl N-[(1S)-1-methyl-2-oxo-2-(phenylmethoxy)ethyl]phosphoramidate];
3′-deoxycytidine 5′-[phenyl N-[(1S)-2-methoxy-1-methyl-2-oxoethyl] phosphoramidate]; and
3′-deoxycytidine 5′-[phenyl N-[(1S)-1-methyl-2-oxo-2-(phenylmethoxy)ethyl] phosphoramidate];
and salts and solvates thereof.

### Processes

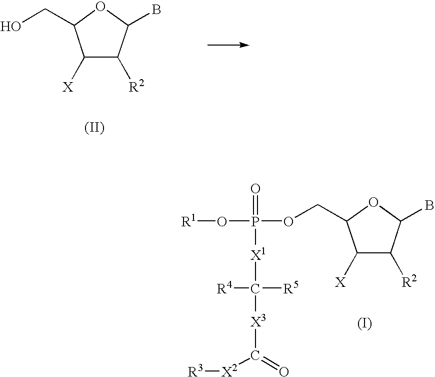

Compounds of Formula (I) and (Ia) may be prepared from compounds of Formula (I) using a reagent R¹O[X¹C(R⁴)(R⁵)X³C(O)X²C(R³)]P(O)Cl in a suitable solvent such as tetrahydrofuran, DMF, or acetonitrile with a suitable base such as pyridine, N-methyl imidazole, or tert-butyl magnesium chloride. Where R² represents hydroxy, and B represents (b), the preferred solvent is a combination of tetrahydrofuran and pyridine, and the preferred base is tert-butyl magnesium chloride used in excess (greater than 2 equivalents).

Compounds of formula R¹O[X¹C(R⁴)(R⁵)X³C(O)X²(R³)]P(O)Cl may be prepared from compounds of formula P(O)Cl₃ by standard methods known in the art (see for example Koszalka, G. W., Daluge, S. M., Boyd, F. L., Annual Rep Med Chem 1998, 33, 163-171 and the references cited therein).

Compounds of formula (II) may be prepared by methods analogous to those known in the art, for example Collect. Czech. Chem. Commun. (1973) 38, 1173-78; Tetrahedron (1998) 54, 13529-46; J. Med. Chem. (1991) 34, 2195; S. Chem. Soc. Chem Commun. (1989) 14, 955-57; G. Gosselin et al, Nucleosides and Nucleotides (1995) 14, 611-617; A. Kumar et al, Nucleosides and Nucleotides (1994) 13, 1049-1057; and T-S Lin et al, J. Med. Chem. (1991) 34, 693-701.

Compounds of Formula (I) and (Ia) in which R² is hydroxy may also be prepared from compounds of Formula (II) by protecting the R² hydroxy group with a suitable protecting agent, for example as an ether (using benzyl ether or silyl ether), or as an ester, then reacting with a reagent R¹O[X¹C(R⁴)(R⁵)X³C(O)X²(R³)]P(O)Cl in a suitable solvent such as tetrahydrofuran, DMF, or acetonitrile with a suitable base such as pyridine, N-methyl imidazole, or tert-butyl magnesium chloride, and finally deprotecting the hydroxy group. Suitable protecting groups may be found, but are not limited to, those described in T W Greene and P G M Wuts ‘Protective Groups in Organic Synthesis’, 3^{rd} edition (1999), J Wiley and Sons.

### Assay

The potential for compounds of the invention to inhibit NS5B wildtype HCV polymerase activity may be demonstrated, for example, using the following cell based assay:

Replicon ELISA

Cells

The 5-15 subline of Huh-7 cells (Lohmann, V., Korner, F., Koch, J-O., Herian, U., Theilmann, L. & Bartenschlager, R., 1999, Science, 2, pp 110-113) were used for these assays. These are human hepatocellular carcinoma cells stably transfected with an HCV replicon comprising the majority of the HCV 1b genome with the addition of a selectable marker gene, but lacking the genes encoding for all structural proteins and non-structural protein (NS) 2. The replicon RNA is self-replicating and fully functional viral proteins are translated from it. A quantifiable and specific reduction of expressed protein in the presence of a drug can be used as a measure of replicon inhibition.

Compounds

Stock solutions of compound samples were formulated to 40 mM in DMSO.

Method

Culture step: 100 μl volumes of assay medium (Dulbecco's Minimal Essential Medium (DMEM) with 4500 mg/L glucose and supplemented with 10% foetal bovine serum, 100 iu/ml penicillin, 1001 μg/ml streptomycin, 2 mM L-glutamine and 1% non-essential amino acids solution) were added to each well of a 96-well tissue culture plate. The 40 mM stock solutions of compound were further diluted in assay medium to twice the highest final concentration required, and 100 μl aliquots were transferred into two wells in the top row of the plate. Serial doubling dilutions were then made down the plate leaving the bottom two rows compound free. A 100 μl volume of Huh-7 5-15 cell suspension of 2×10⁵ cells/ml in assay medium was added to all wells. The plates were incubated at 37° C. in a 5% CO₂ atmosphere for 72 hours.

ELISA step: Growth medium was removed from the plate and the cell monolayers were washed gently once with phosphate buffered saline (PBS) prior to fixing with a 1:1 mix of acetone:methanol for 5 minutes. The plate was washed again with PBS, blotted dry and 100 μl of ELISA diluent (PBS+0.05% Tween 20+2% skimmed milk powder) was added to each well. The plate was incubated at 37° C. for 30 minutes and the diluent removed. Each well, except one row of the compound free wells, then received 50 μl of murine monoclonal antibody, diluted to llg/ml, raised to a non-structural protein, more specifically NS4a. The control row received 50 μl/well of diluent alone. The plate was incubated for 2 hours, the primary antibody was removed and the cell sheets washed thoroughly with PBS+0.05% Tween 20. Rabbit anti-mouse, polyclonal antibody conjugated to horseradish peroxidase was diluted 1/1000 and 50 μl was added to all wells. Following incubation for one further hour, the secondary antibody was removed and the plate was washed thoroughly in PBS/Tween. The plate was blotted dry and 5011 of orthophenylene diamine/peroxide substrate in urea buffer was added to all wells and colour development was allowed to proceed at room temperature. The reaction was stopped by the addition of 25 μl per well of 2M sulphuric acid and the plates were read spectrophotometrically at 490 nm.

The ELISA solutions were removed from the plates, and the cell sheets were washed with water, blotted dry and stained with 5% carbol fuchsin. After 30 minutes the stain was removed and the plates were washed with water and allowed to air dry.

Data Analysis

The absorbance values from all compound-free wells that had received both primary and secondary antibodies were averaged to obtain a positive control value. The mean absorbance value from the compound-free wells that had not received the primary antibody was used to provide the negative (background) control value. The readings from the duplicate wells at each compound concentration were averaged and, after the subtraction of the mean background from all values, were expressed as a percentage of the positive control signal. Grafit software was used to plot the curve of percentage inhibition against compound concentration and derive the 50% inhibitory concentration (IC₅₀) for the compound.

In-assay cytotoxicity was assessed by microscopic examination of the stained cell sheets, and expressed as the lowest compound concentration at which any cellular effect was visible.

Accordingly, the compounds of the invention are of potential therapeutic benefit in the treatment and prophylaxis of HCV.

Thus, there is provided as a further aspect of the present invention a compound of formula (I) or a physiologically acceptable salt or solvate thereof for use in human or veterinary medicine, particularly in the treatment or prophylaxis of viral infection, particularly HCV infection. Compounds of the present invention are also useful in the treatment and/or prophylaxis of viral infection by hepaciviruses, such as GBV-A, GBV-B, GBV-C and HCV, pestiviruses, such as BVDV, and flaviviruses such as West Nile Virus and Yellow Fever Virus.

It will be appreciated that references herein to treatment extend to prophylaxis as well as the treatment of established conditions. It will further be appreciated that references herein to treatment or prophylaxis of HCV infection includes treatment or prophylaxis of HCV-associated disease such as liver fibrosis, cirrhosis and hepatocellular carcinoma.

According to another aspect of the invention, there is provided the use of a compound of formula (I) or a physiologically acceptable salt or solvate thereof for the manufacture of a medicament for the treatment and/or prophylaxis of viral infection, particularly HCV infection.

According to another aspect of the invention, there is provided a compound of formula (I) or a physiologically acceptable salt or solvate thereof use in treating and/or the prophylaxis of a viral infection, particularly HCV infection.

In a further or alternative aspect there is provided a method for the treatment of a human or animal subject with viral infection, particularly HCV infection, which method comprises administering to said human or animal subject an effective amount of a compound of formula (I) or a physiologically acceptable salt or solvate thereof.

The compounds according to the invention may be formulated for administration in any convenient way, and the invention therefore also includes within its scope pharmaceutical compositions for use in therapy, comprising a compound of formula (I) or a physiologically acceptable salt or solvate thereof in admixture with one or more physiologically acceptable diluents or carriers.

There is also provided according to the invention a process for preparation of such a pharmaceutical composition which comprises mixing the ingredients.

The compounds according to the invention may, for example, be formulated for oral, buccal, parenteral, topical or rectal administration.

Tablets and capsules for oral administration may contain conventional excipients such as binding agents, for example syrup, acacia, gelatin, sorbitol, tragacanth, mucilage of starch or polyvinyl pyrrolidone; fillers, for example, lactose, microcrystalline cellulose, sugar, maize-starch, calcium phosphate or sorbitol; lubricants, for example, magnesium stearate, stearic acid, talc, polyethylene glycol or silica; disintegrants, for example, potato starch, croscarmellose sodium or sodium starch glycollate; or wetting agents such as sodium lauryl sulphate. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxymethyl cellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example almond oil, fractionated coconut oil, oily esters, propylene glycol or ethyl alcohol; or preservatives, for example, methyl or propyl p-hydroxybenzoates or sorbic acid. The preparations may also contain buffer salts, flavouring, colouring and/or sweetening agents (e.g. mannitol) as appropriate.

For buccal administration the compositions may take the form of tablets or lozenges formulated in conventional manner.

The compounds may also be formulated as suppositories, e.g. containing conventional suppository bases such as cocoa butter or other glycerides.

The compounds according to the invention may also be formulated for parenteral administration by bolus injection or continuous infusion and may be presented in unit dose form, for instance as ampoules, vials, small volume infusions or pre-filled syringes, or in multi-dose containers with an added preservative. The compositions may take such forms as solutions, suspensions, or emulsions in aqueous or non-aqueous vehicles, and may contain formulatory agents such as anti-oxidants, buffers, antimicrobial agents and/or toxicity adjusting agents. Alternatively, the active ingredient may be in powder form for constitution with a suitable vehicle, e.g. sterile, pyrogen-free water, before use. The dry solid presentation may be prepared by filling a sterile powder aseptically into individual sterile containers or by filling a sterile solution aseptically into each container and freeze-drying.

By topical administration as used herein, we include administration by insufflation and inhalation. Examples of various types of preparation for topical administration include ointments, creams, lotions, powders, pessaries, sprays, aerosols, capsules or cartridges for use in an inhaler or insufflator or drops (e.g. eye or nose drops).

Ointmnents and creams may, for example, be formulated with an aqueous or oily base with the addition of suitable thickening and/or gelling agents and/or solvents. Such bases may thus, for example, include water and/or an oil such as liquid paraffin or a vegetable oil such as arachis oil or castor oil or a solvent such as a polyethylene glycol. Thickening agents which may be used include soft paraffin, aluminium stearate, cetostearyl alcohol, polyethylene glycols, microcrystalline wax and beeswax.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents or suspending agents.

Spray compositions may be formulated, for example, as aqueous solutions or suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluorethane, carbon dioxide or other suitable gas.

Capsules and cartridges for use in an inhaler or insufflator, of for example gelatin, may be formulated containing a powder mix of a compound of the invention and a suitable powder base such as lactose or starch.

The pharmaceutical compositions according to the invention may also be used in combination with other therapeutic agents, for example immune therapies (eg. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (eg N-acetylcysteine), cytokine agonists, cytokine antagonists, lung surfactants and/or antimicrobial and anti-viral agents (eg ribavirin and amantidine). The compositions according to the invention may also be used in combination with gene replacement therapy.

The invention thus provides, in a further aspect, a combination comprising a compound of formula (I) or a physiologically acceptable salt or solvate thereof together with another therapeutically active agent.

The combination referred to above may conveniently be presented for use in the form of a pharmaceutical formulation and thus pharmaceutical formulations comprising a combination as defined above together with a pharmaceutically acceptable carrier thereof represent a further aspect of the invention.

The individual components of such combinations may be administered either sequentially or simultaneously in separate or combined pharmaceutical formulations. Appropriate doses of known therapeutic agents will be readily appreciated by those skilled in the art.

The compound of the invention may conveniently be administered in amounts of, for example, 0.01 to 100 mg/kg body weight, suitably 0.05 to 25 mg/kg body weight orally, one or more times a day. The precise dose will of course depend on the age and condition of the patient, the particular route of administration chosen, and is entirely within the discretion of the administering physician.

The compounds of the invention have useful duration of action.

The following non-limiting Examples illustrate the present invention.

### EXAMPLES

Intermediate 1

4-tert-Butylphenyl phosphodichloridate

A solution containing 4-tert-butylphenol (15.0 g, 0.1 Mol) and triethylamine (13.95 mL) in ether (200 mL) was added dropwise with stirring at 0° C. over 2 h to a solution of phosphoryl chloride (11.1 mL) in ether (100 mL). The cooling bath was removed and the mixture was stirred for a further 18 h. The mixture was filtered and volatiles were removed from the filtrate to give a pale yellow oil which was distilled to give the title compound as a colourless oil (18.72 g).

NMR: (CDCl₃) δ 7.43 (d, J=8.5 Hz, 2H); 7.20 (dd, J=8.5, 2.25 Hz, 2H); 1.32 (s, 9H).

Intermediate 2

L-Alanine, N-(chlorophenoxyphosphinyl)methyl Ester

A suspension of L-alanine methyl ester hydrochloride (1.00 g, 7.2 mMol) in dichloromethane (10 mL) was treated with phenyl phosphorodichloridate (1.07 g, 5.1 m Mole). The mixture was stirred under nitrogen and cooled to −78° C. Di-isopropylethylamine (2.5 mL) was added in portions over 30 min. The cooling bath was removed and the mixture was stirred at room temperature overnight. Volatiles were removed in vacuo. The residue was extracted with ether (25 mL) and the extracts were concentrated to give the title compound as a colourless oil (1.25 g), used with no further purification.

NMR: (CDCl₃) δ 7.1-7.4 (m, 5H); 4.0-4.3 (m, 10); 3.71+3.72 (2x s, total 3H); 1.53+1.39 (2x m, 3H total).

Intermediate 3

L-Alanine, N-(chlorophenoxyphosphinyl)phenylmethyl Ester

The title compound was prepared as an oil according to the procedure described for Intermediate 2, using L-alanine benzyl ester hydrochloride and phenyl phosphorodichloridate.

NMR: (CDCl₃) δ 7.1-7.4 (m, 10N); 5.20+5.22 (2x s, 2H); 4.9-5.1 (m, 1H); 1.51+1.52 (2x d, 3H total).

Intermediate 4

L-Alanine, N-(chloro-4-tert-butylphenoxyphosphinyl)phenylmethyl Ester

The title compound was prepared as a colourless oil according to the procedure described for Intermediate 2, using L-alanine benzyl ester hydrochloride and Intermediate 1.

NMR: (CDCl₃) δ 7.32-7.40 (m, 7H); 7.15 (m, 2H); 5.21 (m, 21); 4.18-4.36 (m, 2H); 1.52 (2, 3H); 1.30 (s, 9H).

### Example 1

3′-Deoxyguanosine-5′-[4-(1,1-dimethylethyl)phenyl-N-[(1S)-1-methyl-2-oxo-2-(phenylmethoxy)ethyl]phosphoramidate]

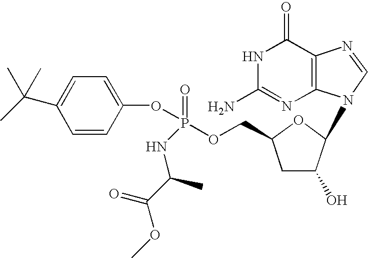

A suspension of 3′-deoxyguanosine (27 mg) in pyridine (1 mL) was stirred under nitrogen and treated with 11.0M tert-butyl magnesium chloride in tetrahydrofuran (220 μL). The resulting solution was stirred at 20° C. for 1 h and then treated with a solution of Intermediate 4 (45 mg) in tetrahydrofuran (0.8 mL). The mixture was stirred for a further 18 h and then evaporated to dryness. The residue was partitioned between water (10 mL) and ethyl acetate (25 mL). The organic phase was collected and dried (MgSO₄). Removal of solvent gave a colourless gum which was purified on silica gel preparative plates with 8:1 (v:v) dichloromethane:methanol affording the title compound as a solid.

Mass spec (electrospray) m/z calcd for (C₃₀H₃₇N₆O₈P+H)⁺: 641.

Found: (M+H)⁺=641.

### Example 2

3′-Deoxycytidine-5′-[phenyl-N-[(1S)-2-methoxy-1-methyl-2-oxoethyl]phosphoramidate]

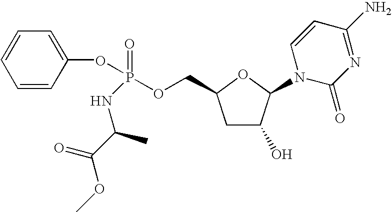

A suspension of 3′-deoxycytidine (25 mg) in pyridine (1 mL) was stirred under nitrogen at −10° C. and treated with 11.0M tert-butyl magnesium chloride in tetrahydrofuran (260 μL). The resulting mixture was stirred for a further 20 rin and then treated with a solution of Intermediate 2 (37 mg) in pyridine (0.6 mL). The mixture was stirred at 0° C. for 4 h and then stored at 0° C. for 18 h. Solvent was removed under reduced pressure. The residue was purified on silica gel preparative plates with 8:1 (v:v) dichloromethane:methanol affording the title compound as a solid.

Mass spec (electrospray) m/z calcd for (C₁₉H₂₅N₄O₈P+H)⁺: 469.

Found: (M+H)⁺=469.

### Example 3

3′-Deoxycytidine-5′-[phenyl-N-[(1S)-1-methyl-2-oxo-2-(phenylmethoxy)ethyl]phosphoramidate]

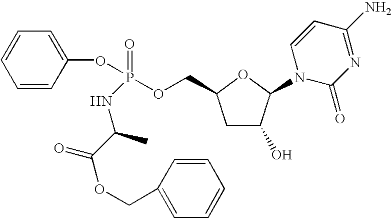

To a suspension of 3′-deoxycytidine (25 mg, 0.11 mMol) in dry pyridine (1 ml) was added tert-butylmagnesium chloride (1.0M solution in THF, 352 μl, 0.35 mMol) giving an orange suspension which was stirred at ambient temperature under nitrogen for 1 h. Intermediate 3 (0.14 nmMol, 47.8 mg) in dry THF (1 ml) was added and the reaction mixture was stirred for a further 3.5 h. The reaction was then quenched with methanol (1 ml) and the volatiles were evaporated in vacuo. The residue was partitioned between ethyl acetate and water and the organics were dried (MgSO₄), filtered and evaporated to give a white solid (29.5 mg). The crude product was purified on silica gel preparative plates with 9:1 (v:v) dichloromethane:methanol affording the title compound as a white solid.

Mass spec (electrospray) m/z calcd for (C₂₅H₂₉N₄O₈P+H)⁺: 545.

Found: (M+W)⁺=545.

Biological Data

The compounds of Examples 1-3 were tested in the in vitro tests described earlier. All the compounds had an IC₅₀ value of <200 μM in the HCV replicon assay.

1. Compounds of formula (I) 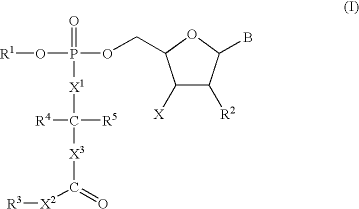
wherein X represents H, F, N₃, NH₂, —CN, or —OMe;
X¹ represents O or NR⁷;
X² represents O, NH, NR⁶ or S, or when X³ is O then X² is absent;
X³ is absent, or when X¹ is O then X³ represents O;
R¹ represents hydrogen; optionally substituted C₁₋₆alkyl; optionally substituted aryl; or optionally substituted heteroaryl;
R² represents hydroxy, OCOR⁶, or OCO₂R⁶;
R³ represents H, optionally substituted C₁₋₆alkyl, optionally substituted aryl, optionally substituted heteroaryl or optionally substituted heterocyclyl;
R⁴ and R⁵ are independently selected from hydrogen, optionally substituted C₁₋₆alkyl, optionally substituted aryl, or optionally substituted aralkyl;
R⁶ represents optionally substituted C₁₋₆alkyl or optionally substituted aryl;
R⁷ represents H, optionally substituted C₁₋₆alkyl, or optionally substituted aryl, wherein when R⁴ and R⁷ are each alkyl they may be linked to form a 5- or 6-membered ring;
B represents (a), (b), (c), or (d) 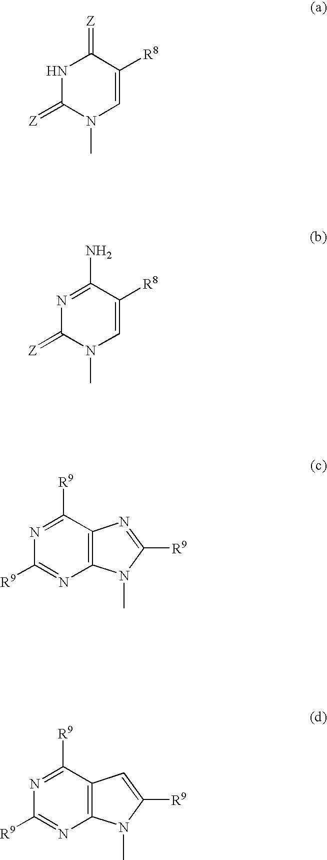
wherein Z represents O or S;
R⁸ represents H, halo, C₂₋₄alkynyl, trifluoromethyl, C₁₋₃alkoxy, hydroxy, methylthio, amino, nitro, or C₁₋₃alkyl wherein the C₁₋₃alkyl may be optionally substituted by hydroxy, halo, amino, or OR¹⁰ wherein R¹⁰ represents C₁₋₆alkyl optionally substituted by aryl which may itself be optionally substituted; and R⁹ represents H, halo, hydroxy, OR⁶, SR⁶ or NR³R³;
and salts and solvates thereof.

2. Compounds of formula (I) as claimed in claim 1, represented by formula (Ia) 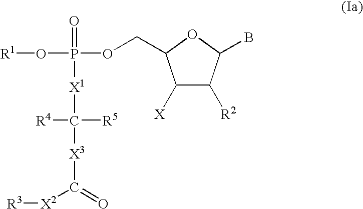
wherein X represents H, F, N₃, NH₂, —CN, or —OMe;
X¹ represents O or NR⁷;
X² represents O, NH, NR⁶ or S, or when X³ is O then X² is absent;
X³ is absent, or when X¹ is 0 then X³ represents O;
R¹ represents hydrogen; optionally substituted aryl; or optionally substituted heteroaryl;
R² represents hydroxy, OCOR⁶, or OCO₂R⁶;
R³ represents H, optionally substituted C₁₋₆alkyl, optionally substituted aryl, optionally substituted heteroaryl or optionally substituted heterocyclyl;
R⁴ and R⁵ are independently selected from hydrogen, optionally substituted C₁₋₆alkyl, optionally substituted aryl, or optionally substituted aralkyl;
R⁶ represents optionally substituted C₁₋₆alkyl or optionally substituted aryl;
R⁷ represents H, optionally substituted C₁₋₆alkyl, or optionally substituted aryl, wherein when R⁴ and R⁷ are each alkyl they may be linked to form a 5- or 6-membered ring;
B represents (a), (b), (c), or (d) 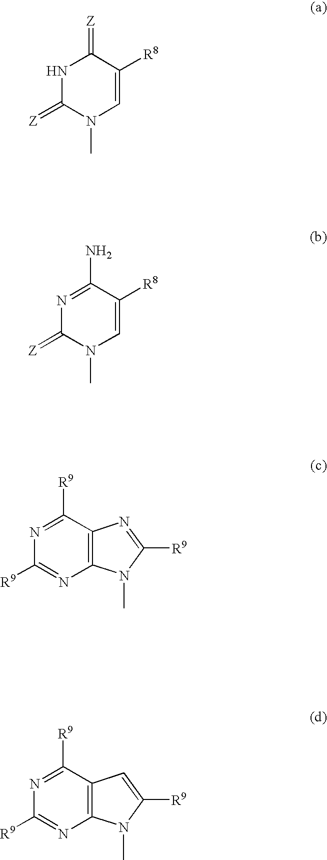
wherein Z represents O or S;
R⁸ represents halo, C₂₋₄alkynyl, trifluoromethyl, C₁₋₃alkoxy, hydroxy, methylthio, amino, nitro, or C₁₋₃alkyl wherein the C₁₋₃alkyl may be optionally substituted by hydroxy, halo, amino, or OR¹⁰ wherein R¹⁰ represents C₁₋₆alkyl optionally substituted by aryl which may itself be optionally substituted; and
R⁹ represents H, halo, hydroxy, OR⁶, SR⁶ or NR³R³;
and salts and solvates thereof.

3. (canceled)

4. (canceled)

5. (canceled)

6. A method for the treatment of a human or animal subject with viral infection, which method comprises administering to said human or animal subject an effective amount of a compound of formula (I) as claimed in claim 1.

7. A method as claimed in claim 6, wherein the viral infection is HCV infection.

8. A process for the preparation of a compound of Formula (I) as claimed in claim 1, comprising reaction of a compound of Formula (II) 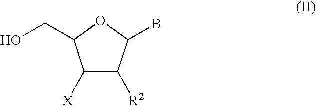
wherein B, X and R² are as described in Formula (I), with a reagent R¹O[X¹C(R⁴)(R⁵)X³C(O)X²(R³)]P(O)Cl, wherein R¹, R³-R⁵ and X¹-X³ are as described in Formula (I), in a suitable solvent with a suitable base.

9. A pharmaceutical formulation comprising a compound of Formula (I) as claimed in claim 1 together with a pharmaceutically acceptable diluent or carrier therefor.
